(19) United States
(12) Patent Application Publication
(10) Pub. No.: US 2010/0076275 A1
(43) Pub. Date: Mar. 25, 2010

(54) System and Method for Remote Healthcare Monitoring

(76) Inventors: Edmond Chu, Erie, CO (US); Robert Graham Buchanan, Broomfield, CO (US); John A. Van Tol, Saratoga, WY (US); Eric J. Norgard, Louisville, CO (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/508,830

(22) Filed: Jul. 24, 2009

Related U.S. Application Data
(60) Provisional application No. 61/083,465, filed on Jul. 24, 2008.

(51) Int. Cl. A61B 5/00

(52) U.S. Cl. 600/300

(57) ABSTRACT

A system and method for remote health monitoring is described. One embodiment includes a portable health monitoring device comprising a data transfer interface configured to receive a protocol, wherein the protocol comprises branching logic, a display configured to present a user with at least one question from the branching logic, a user interface configured to receive information from the user, a diagnostic device interface configured to communicate with a diagnostic device, a memory configured to store data related to the user, and a processor configured to analyze at least a portion of the data related to the user, wherein the processor is further configured to transmit at least a portion of the data related to the user to a monitoring location via the data transfer interface.

### PRIORITY

The present application claims priority from to commonly owned and assigned application No. 61/083,465, Attorney Docket No. NSPI-010/00US 307782-2046, entitled SYSTEM AND METHOD FOR REMOTE HEALTHCARE MONITORING, which is incorporated herein by reference.

### FIELD OF THE INVENTION

The present invention relates to systems and methods for remote healthcare monitoring. Embodiments of the invention relate generally to systems and methods which permit a portable health monitoring device to collect, analyze and transmit user health information to a monitoring location.

### BACKGROUND OF THE INVENTION

There are many problems and issues that arise when monitoring the health of an individual, especially sick individuals and clinical trial subjects. For example, new drug trials often require clinical trial subjects (drug trial participants) to maintain a “paper” diary that provides a record of each participant's condition over time. Information collected could include measurement results from health monitoring/diagnostic devices (typically simple devices that a participant can operate) and responses/answers to a set of pre-defined questions at time intervals defined by the drug trial procedure. There are a number of deficiencies associated with the use of paper diary for clinical trial data collection, including:
a) the process of transposing measurement results from health monitoring/diagnostic devices to paper are prone to errors;
b) the time of data collection cannot be assured (for example, a clinical trial subject may have filled out two weeks of responses in the parking lot before returning the paper diary); and
c) it is difficult to implement branching logic, which helps to both reduce the amount of time a clinical trial subject has to spend answering questions and gets more complete responses on important issues, in the questionnaire.

Some of the deficiencies of paper diaries have been addressed by electronic diaries through the use of computers or smart devices. For example, software on electronic diaries can add date- and time-stamps to the recorded data and provide branching logic capabilities to questionnaires. In some implementations, a diagnostic device can also be electronically linked to the electronic diary device allowing direct transfer of measurement results. However, the current art of electronic diaries treats monitoring/measurement and questionnaire as independent processes, fails to provide a mechanism for adaptive trials and lack individualized device setups that may be required for certain users or certain trials. Accordingly, a new system and method are needed.

### SUMMARY OF THE INVENTION

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

The present invention relates to systems and methods for remote health monitoring. In one exemplary embodiment, the present invention can include a portable health monitoring device comprising a data transfer interface configured to receive a protocol, wherein the protocol comprises branching logic, a display configured to present a user with at least one question from the branching logic, a user interface configured to receive information from the user, a diagnostic device interface configured to communicate with a diagnostic device, a memory configured to store data related to the user, and a processor configured to analyze at least a portion of the data related to the user, wherein the processor is further configured to transmit at least a portion of the data related to the user to a monitoring location via the data transfer interface. The portable health monitoring device may further comprise a removable flow meter for spirometric measurements. In some embodiments, diagnostic device data from the removable flow meter may be immediately used for user diagnostics and branching logic. The portable health monitoring device is further configured to receive protocol updates via the data transfer interface to update the operation of the portable health monitoring device.

In another exemplary embodiment, the present invention can include a system for remotely monitoring healthcare patients comprising a portable health monitoring device, wherein the portable health monitoring device is configured to collect, analyze and store health information of a healthcare patient and remotely receive a protocol update, wherein the protocol update modifies a protocol for collecting and analyzing the health information, a health information data store for storing the health information, and a central monitoring server coupled to the health information data store, the central monitoring server capable of receiving health information from the portable health monitoring device, the central monitoring device further capable of analyzing the health information.

In another exemplary embodiment, the present invention can include a method for collecting user health information, comprising loading a protocol on a portable health monitoring device, wherein the protocol contains instructions for collecting and analyzing the user health information, providing the portable health monitoring device to a user, receiving a protocol update at the portable health monitoring device, wherein the protocol update modifies the protocol, collecting the user health information using the portable health monitoring device, transmitting the user health information from the portable health monitoring device to a monitoring location.

As previously stated, the above-described embodiments and implementations are for illustration purposes only. Numerous other embodiments, implementations, and details of the invention are easily recognized by those of skill in the art from the following descriptions and claims.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings, wherein:

FIG. 1 is a high level diagram of an embodiment of a system in accordance with the present invention.

FIG. 2A is an embodiment of a portable health monitoring device in accordance with the present invention.

FIG. 2B is an embodiment of a portable health monitoring device in accordance with the present invention.

FIG. 3 shows a block diagram of an exemplary mobile electronics unit that may be used in accordance with the present invention.

FIG. 4 is a block diagram of an exemplary system in accordance with the present invention.

FIG. 5 is a block diagram of an exemplary system in accordance with the present invention.

FIG. 6 depicts a branching logic flow chart consistent with the present invention.

FIG. 7 shows a mobile solution data flow diagram consistent with the present invention.

FIG. 8 depicts exemplary user interface screens that could be used with the present invention.

### DETAILED DESCRIPTION

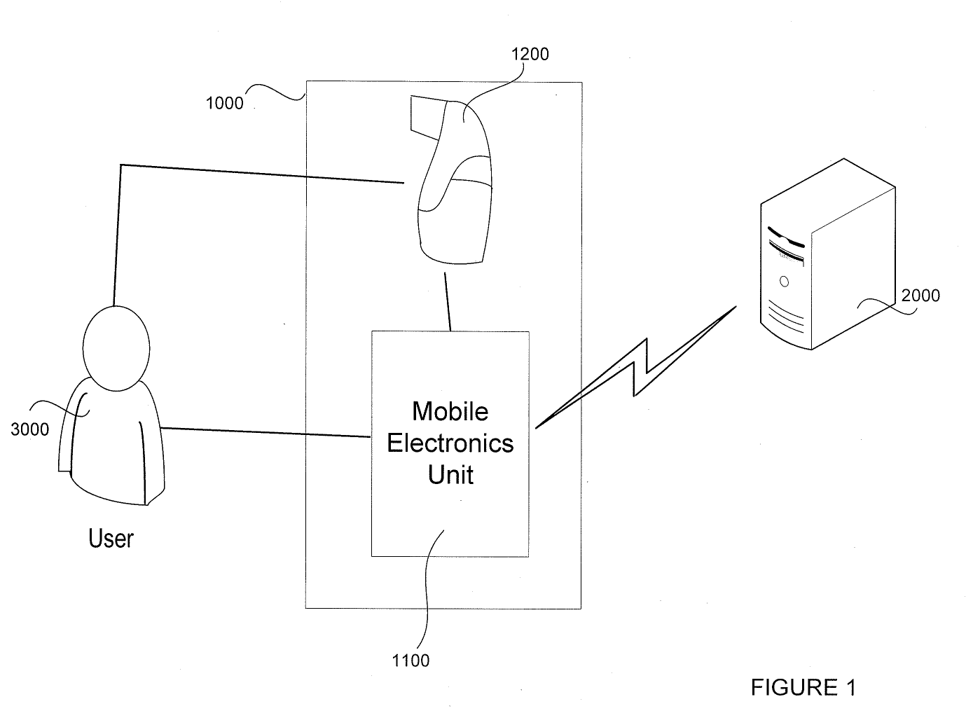

Referring now to the drawings, where like or similar elements are designated with identical reference numerals throughout the several views, FIG. 1 is a high level diagram of an embodiment of a system constructed in accordance with the present invention. As shown, FIG. 1 includes a portable health monitoring device 1000, a monitoring location 2000 and a user 3000. The portable health monitoring device 1000 in FIG. 1 includes a removable flow meter 1200 and a mobile electronics unit 1100. The user 3000 can interact with one or both of the removable flow meter 1200 and the mobile electronics unit 1100. The portable health monitoring device 1000 can also communicate with a monitoring location 2000. The monitoring location 2000 could be a server, collection of servers, an administrator computer, or other device configured to receive and store data from the portable health monitoring device 1000. For example, the monitoring location could include a single location or multiple locations used by a monitoring party (healthcare provider, clinical trial investigator, device information technology/services technician, etc.) to monitor the user's 3000 health data. In another embodiment, the monitoring location 2000 could be a server that is limited to collecting information from one or more portable health monitoring devices 1000.

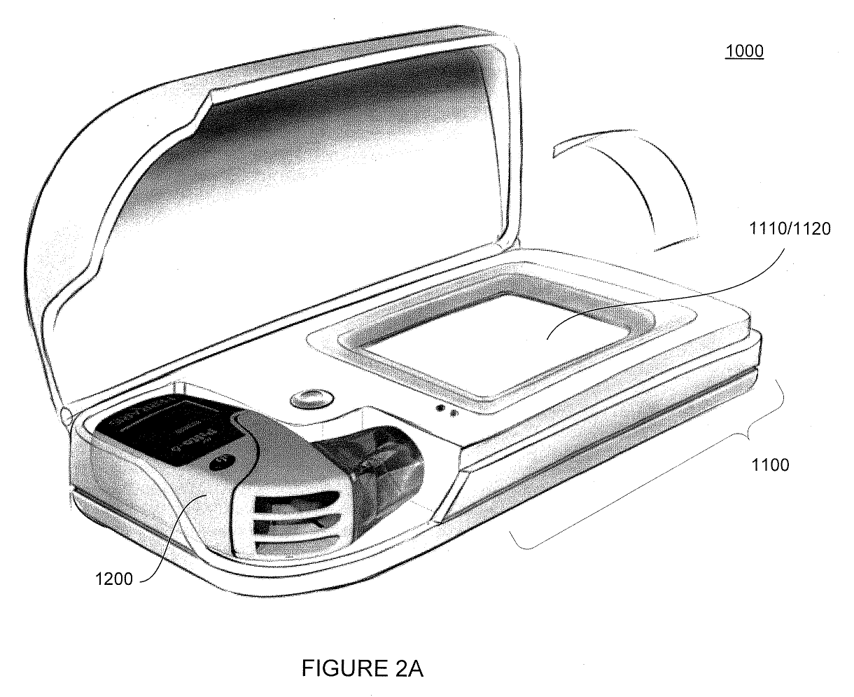

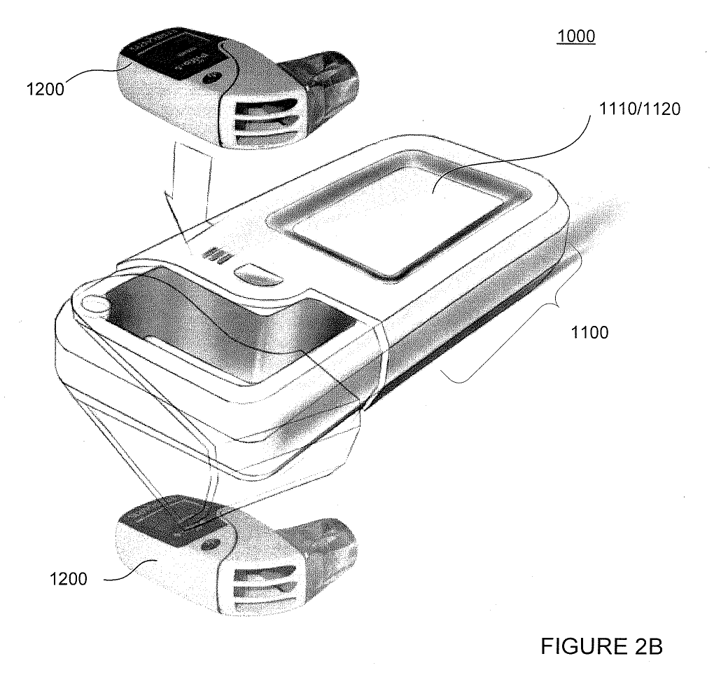

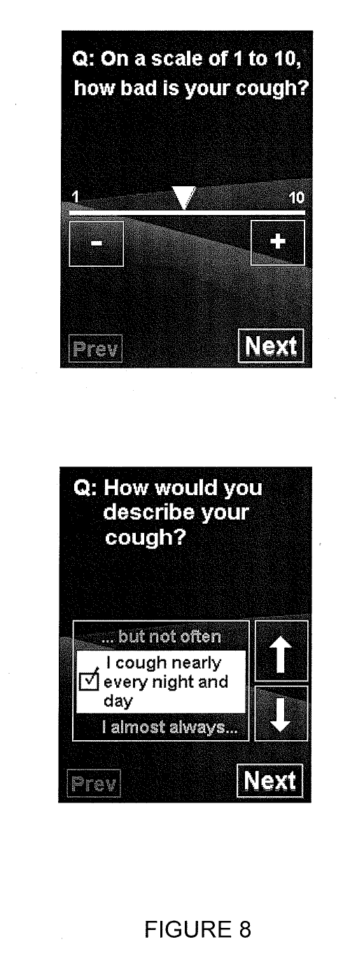

FIG. 2A and FIG. 2B (collectively FIG. 2) show an embodiment of a portable health monitoring device 1000 in accordance with the present invention. In this embodiment, the portable health monitoring device 1000 includes a mobile electronic unit 1100 and a removable electronic flow meter 1200. The mobile electronic unit 1100 can serve as both an electronic diary and a data gateway for receiving information from, and transmitting information to, a monitoring location 2000. In FIG. 2 the mobile electronic unit 1100 is shown to include a display 1110 for displaying instructions, questions, results and other information to a user. In this embodiment, the display 1110 is a touch screen which can serve as both the display 1110, for displaying information to a user, and a user interface 1120, for receiving information from the user. For example, FIG. 8 shows exemplary screens which may be used on a touch screen to make the display 1110 also serve as a user interface 1120. In another embodiment, the display 1110 and user interface 1120 could be separate. For example, the display 1110 may comprise a liquid crystal display (LCD), while the user interface 1120 may comprise a button keypad. Those of skill in the art will realize numerous modifications and options consistent with the present invention.

Along with the display 1110 the mobile electronics unit 1100 could also be configured to include a speaker, headphone jack, or other audio device for communicating audio signals to a user. This can be incorporated as part the display 1110, or as a separate structure. Such an audio device could be used as a means for communicating with visually impaired users, or just as an alternative means of communicating information, such as instructions, feedback, and questions, to a user. The display 1110 and the user interface 1120 could also be configured for use by an impaired user, such as visually impaired or physically impaired user. For example, the display 1110 could include a Fresnel lens for users with a visual impairment, or the display 1110 could be modified to increase font size for easier readability. The user interface 1120 could also be configured to assist with users with visual or physical impairments—such as elderly users who may have difficulty both seeing and pushing buttons on a small keypad. In one embodiment a touch screen display and user interface could be used where the touch screen includes options for larger text and selections zone to allow for easier readability and selectability.

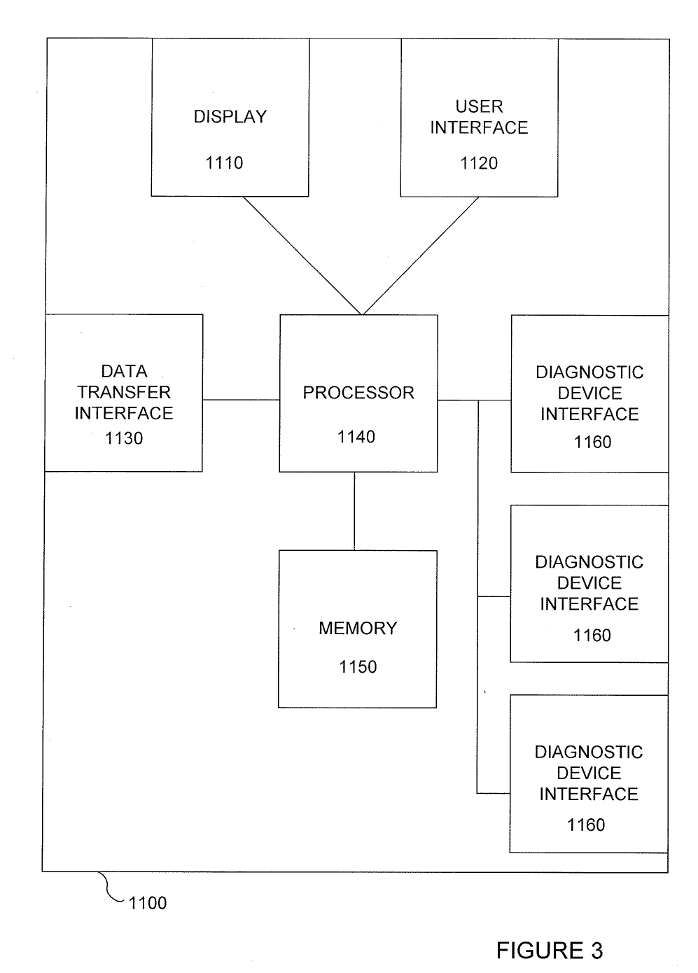

FIG. 3 shows a block diagram for the mobile electronics unit 1100. As shown, the mobile electronics unit 1100 includes a data transfer interface 1130, a processor 1140, a memory 1150 and one or more diagnostic device interfaces 1160. The data transfer interface 1130 may be a cellular interface, a Wi-Fi interface, an Ethernet interface, or some other type of wired or wireless interface for communicating between the mobile electronics unit 1100 and a monitoring location 2000 (not shown in FIG. 3). Those of skill in the art will readily realize that the data transfer interface 1130 may comprise multiple interfaces of the same or different type.

The data transfer interface 1130 can be configured to both receive communications from a monitoring location 2000 and send communications to the monitoring location 2000. For example, the data transfer interface 1130 is capable of receiving a protocol, protocol updates, messages, alerts and other communications from the monitoring location 2000. A protocol is a set of instructions for the mobile electronics unit 1100 which control aspects of how the mobile electronics unit 1100 operates. This may include the questions and branching logic that are provided to the user, the diagnostic information used to process data received from the user, language information, instructions on how to operate the portable health monitoring device 1000, instructions on how to conduct certain health tests, incentive options to encourage proper and timely usage and other forms of compliance, and other information. The use of protocols and protocol updates allow for the same portable health monitoring device 1000 to be more easily and quickly used for various purposes such as different clinical trials and/or health monitoring of users with various health issues. Even with a drug trial, for example, protocol and protocol updates can allow for faster and simpler adaptive trials to be executed. If based on early trial results a certain control group becomes less important or irrelevant, protocol and protocol updates can be used to immediately reassign users within that control group. If the entire protocol does not need to be changed, protocol updates may be used to make more minor variations to the protocol. For example, if based on early user responses a certain trend or issue starts to emerge, the protocol updates may be used to automatically and quickly update the portable health monitoring device(s) 1000 with new tests or questions for users.

Beyond receiving a protocol, the data transfer interface 1130 can be used for receiving message, alerts, device update and other communications. For example, a monitoring party (healthcare provider, clinical trial investigator, device information technology/services technician, etc.) may need to send a specific communication to a user, or a specific update to a particular portable health monitoring device 1000. Messages could include changes in treatment (e.g., changes in dosage, changes in lifestyle factors), reminders for scheduled or unscheduled office visits, or particular questions intended for a specific user. Device updates may include patches or device updates allowing the mobile electronics unit 1100 to operate with a new type or model of diagnostic device. Moreover, given the potentially highly confidential nature of data stored on the portable health monitoring device 1000, the data transfer interface can be configured to receive wiping commands from a monitoring location if the device is lost or stolen. Those of skill in the art will be readily aware of other communications that could be received by the data transfer interface 1130 consistent with the present invention.

In addition to the data transfer interface 1130, the mobile electronics unit can also receive information using the diagnostic device interface(s) 1160 and the user interface 1120. As discussed above, the user interface 1120 can be a touch screen, keypad, biometric device (such as for user identification), or other interface (including a microphone which would allow voice recognition software to be used) to acquire information from a user. The information from the user can include user related information, such as identifying information (password, PIN, name etc.) and health information (responses to health related questions, journal entries, drug usage information), and other information.

The diagnostic device interface 1160 can receive information from a diagnostic device, such as a removable flow meter 1200, blood sugar monitor, pulse oximeter, electrocardiogram, heart rate monitor, blood pressure monitor, thermometer, etc. Diagnostic device may also include devices that obtain environmental conditions related to the user, such as an altimeter, air quality sensor, GPS device, etc. Each diagnostic device may have its own type of diagnostic device data that is communicated to the mobile electronics unit 1100 through the diagnostic device interface 1160. It should be noted that, consistent with the present invention, a diagnostic device could be built into the mobile electronics unit 1100. Moreover, while FIG. 3 shows multiple diagnostic device interfaces 1160, this is not to suggest that multiple diagnostic device interfaces 1160 are required. A single diagnostic device interface 1160 could be used to communicate with one diagnostic device or multiple different diagnostic devices.

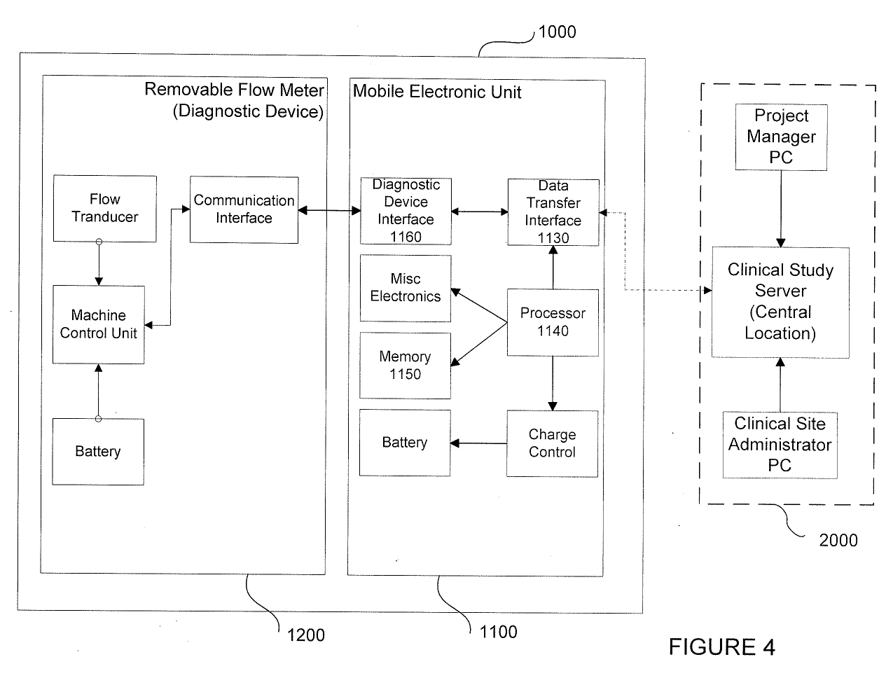

The removable electronic flow meter 1200 is one type of diagnostic device which may be used in the present invention. In one embodiment, the removable electronic flow meter 1200 could be a diagnostic device used for spirometric measurements. As shown in FIG. 2, the removable electronic flow meter 1200 can be stored in the portable health monitoring device 1000. This allows for easy and convenient transportation, use and storage. The storage spot for the removable electronic flow meter 1200 may include a diagnostic device interface 1160 in order to transfer data between the removable electronic flow meter 1200 and the mobile electronic unit 1100. In FIG. 4, it shows a block diagram of an embodiment where the removable electronic flow meter 1200 is configured to communicate with the mobile electronic unit 1100 while docked within the portable health monitoring device 1000.

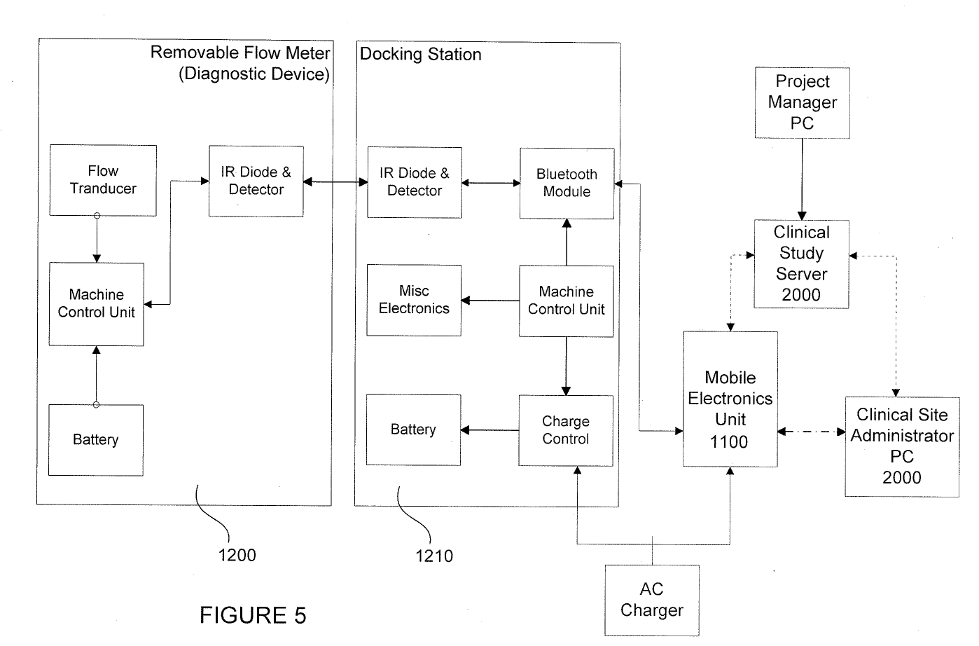

In another embodiment, the removable electronic flow meter 1200 may have a separate docking station 1210 that can interface with the mobile electronic unit 1100. Such a docking station may use a wired interface, such as a USB connection, or a wireless interface, such as a Bluetooth® interface, to enable data transfer between the flow meter docking station 1210 and the mobile electronic unit 1100. In FIG. 5 it depicts a block diagram of a removable flow meter connected to a docking station 1210. The docking station 1210 is further connected to a mobile electronics unit 1100 which is configured to transmit data to a monitoring location 2000. Because the removable electronic flow meter 1200 may not be able to immediately transfer data to the mobile electronic unit 1100, the removable electronic flow meter 1200 can include memory for storing data until transfer is possible. This data may be date- and time-stamped in order to know when the data was acquired. The removable electronic flow meter 1200 may also be replaceable so that the mobile electronics unit 1100 may be reused with various users and various removable electronic flow meters 1200. The use of a removable flow meter 1200 as the diagnostic device is exemplary only. The removable flow meter 1200 may be replaced with numerous other diagnostic devices or used in conjunction with one or more other diagnostic devices.

Information and data received by the mobile electronics unit 1100 can be stored in memory 1150. In FIG. 3, memory 1150 can represent one or more memory structures used to store various information and data for the mobile electronics unit's 1100 operation. For example, information and data related to the user may be stored in non-volatile random access memory (NVRAM) to ensure that data collected using the portable health monitoring device 1000 is not lost if connectivity or data transfer becomes unavailable for an extended period of time. Protocols, protocol updates, and other information which relate to operation of the mobile electronics unit 1100 could be stored in the same NVRAM or in a separate NVRAM structure. Other types of device information, such as firmware, could be stored in read only memory. The mobile electronics unit 1100 may be preprogrammed with basic software which enables usage for receiving and loading a protocol, messages, alerts and protocol updates. Such software may be stored in ROM or RAM. Those of skill in the art will realize many modifications consistent with the present invention.

In use, the data received by the mobile electronics unit 1100 can be date- and time-stamped for storage in memory 1150. The diagnostic device data may have been previously date- and time-stamped by the diagnostic device or, if the diagnostic device is communicating real-time with the mobile electronics unit 1100, the mobile electronics unit 1100 could date- and time-stamp the diagnostic device data. In some embodiments, it may be preferred to transmit data to the monitoring location as soon as possible and potentially as the data is generated. However, if the portable health monitoring device 1000 is unable to communicate with the monitoring location 2000, the data must be stored until connectivity is restored. Additional data stored in the mobile electronics unit 1100 could be data that results from diagnostics or other data processing performed by the processor 1140.

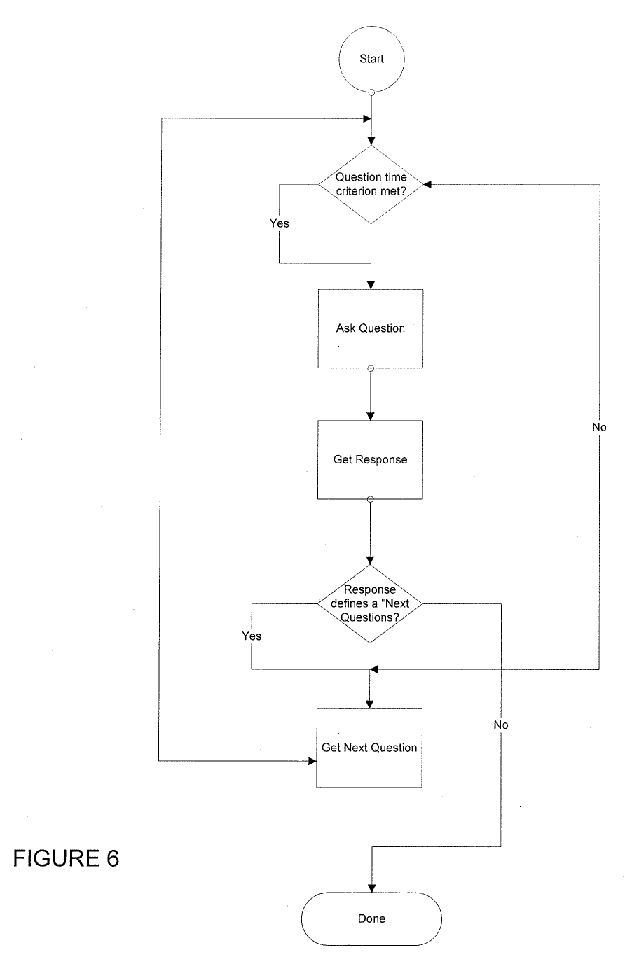

The processor 1140 shown in FIG. 3 may be used to control the collection, analysis and transmission of user data. For example, in an exemplary embodiment the processor 1140 can present the user with questions in accordance with branching logic from a protocol. After a user response is received through a user interface 1120, the processor can analyze the response based on the branching logic in order to select the next question to present to the user. FIG. 6 shows a simple branching logic flow chart consistent with the present invention. As shown in FIG. 6, the branching logic can not only consider user responses in determining whether to present a next question, but the branching logic can also consider other factors, such as time, in determining whether or not to continue.

As the mobile electronics unit 1100 acquires data related to the user, the processor 1140 can analyze the data by running diagnostics or smart algorithms which can alert the user and/or a monitoring party of pending exacerbation or problems for the user or other abnormalities. The portable health monitoring device 1000 may also identify the need for an early office visit, adjustments to drug usage, as well as other functionality. For example, the processor 1140 could automatically acknowledge receipt of protocols, protocol updates, alerts and messages and seek acknowledgement from the user that messages, alerts or other communications have been reviewed and are understood.

The processor 1140 could further control incentive options in accordance with a protocol. Incentive options can include many options depending on the intended user. Generally speaking, incentive options are intended to encourage user compliance and adequate responses. For example, in some cases the display 1110 and user interface 1120 may only be available during a pre-programmable time window to help require users to use the portable health monitoring device at the proper times. In an embodiment, incentive options could include graphic displays directed toward the user's demographic where the graphic display is only received if questions are answered completely and timely. In one embodiment, incentive options could include a points system which allows a user to accumulate points, which can be later exchanged for prizes, based on adequacy (timeliness, completeness, etc.) of responses.

Referring back to the data transfer interface 1130, in addition to receiving communications, it can also be used to transmit information from the portable health monitoring device 1000 to a monitoring location 2000. As the processor 1140 receives, analyzes, and creates data related to the user, the processor 1140 can send this information to the data transfer interface 1130 so that it can be communicated to a monitoring location 2000. The processor 1130 may be programmed to automatically transmit data related to the user as soon as the data is received/generated. If the data transfer interface 1130 has sufficient connectivity, whether to a cellular network, Wi-Fi network, Ethernet connection, etc., the data transfer interface 1130 can alert the processor which can then initiate the transfer.

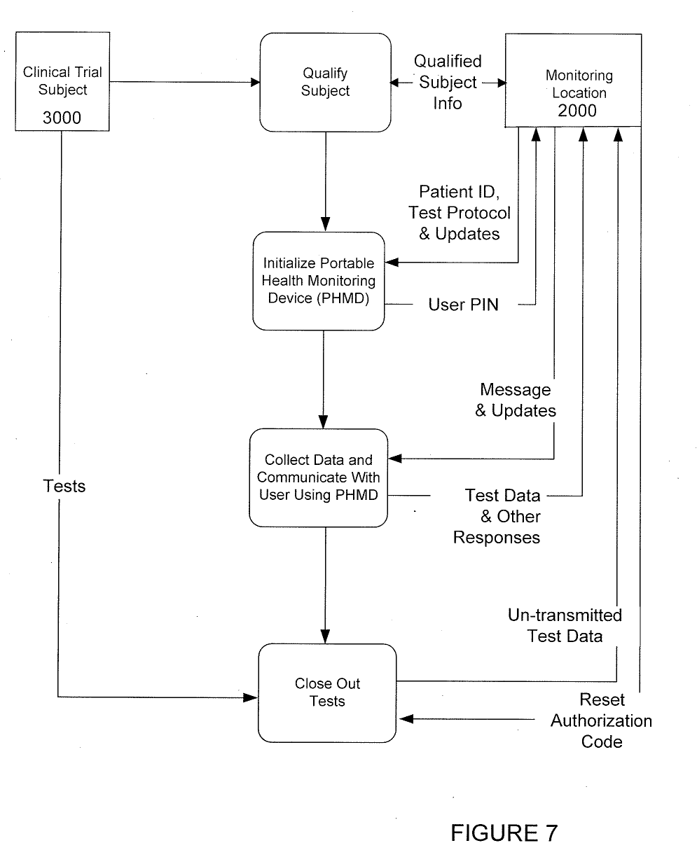

Now referring to FIG. 7 it shows a mobile solution data flow diagram consistent with an embodiment of the present invention. In FIG. 7 a user (in this case a clinical trial subject) is qualified for participation in a clinical trial. The qualified subject's information is sent to a monitoring location where it is used to generate a patient ID, protocol and updates which are transmitted to a portable health monitoring device 1000. Once the portable health monitoring device is initialized with the protocol and other information it can be used by the clinical trial subject. In FIG. 7, it shows that part of the initialization process may include the user selecting a PIN for use with the personal health monitoring device 1000. As the clinical trial subject uses the device and performs certain tests, the portable health monitoring device 1000 collects the user related data and communicates it back to the monitoring location 2000. During the trial period, other messages and updates may be sent to the portable health monitoring device 1000 in order to refine the trial process or seek additional information from the clinical trial subject 3000. Once testing is complete, the portable health monitoring device 1000 can transmit any un-transmitted test data. The monitoring location 2000 can then send a reset authorization code, or wipe command, to the portable health monitoring device 1000 so that any confidential information is deleted and the device is ready to be returned, and then reissued and initialized for the next user.

In conclusion, the present invention provides, among other things, a system and method for remote healthcare monitoring. Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications, and alternative constructions fall within the scope and spirit of the disclosed invention as expressed in the claims.

What is claimed is:

1. A portable health monitoring device comprising:
a data transfer interface configured to receive a protocol, wherein the protocol comprises branching logic;
a display configured to present a user with at least one question from the branching logic;
a user interface configured to receive information from the user;
a diagnostic device interface configured to communicate with a diagnostic device;
a memory configured to store data related to the user; and
a processor configured to analyze at least a portion of the data related to the user, wherein the processor is further configured to transmit at least a portion of the data related to the user to a monitoring location via the data transfer interface.

2. The portable health monitoring device of claim 1, further comprising:
a removable electronic flow meter for spirometric measurements.

3. The portable health monitoring device of claim 1, wherein the processor is further configured to analyze diagnostic device data as part of the branching logic.

4. The portable health monitoring device of claim 1, wherein the data transfer interface is a cellular data transfer interface.

5. The portable health monitoring device of claim 1, wherein the data transfer interface is further configured to receive messages from a monitoring party.

6. The portable health monitoring device of claim 1, wherein the protocol further comprises incentives to encourage timely and complete use.

7. The portable health monitoring device of claim 1, wherein the protocol further comprises diagnostics, wherein the diagnostics provide instructions for the processor to analyze at least a portion of the data related to the user.

8. The portable health monitoring device of claim 1, wherein the display is configured for use by the user where the user is impaired.

9. The portable health monitoring device of claim 1, wherein the user interface is configured to receive the information from the user, wherein the information includes an at least one response from the user, wherein a date and a time of the response is tracked.

10. The portable health monitoring device of claim 1, wherein the diagnostic device interface is selected from a group consisting of a Bluetooth® interface, a USB interface, an IEEE 1394 interface, an IEEE 1284 interface.

11. The portable health monitoring device of claim 1, wherein the data related to the user is based on the information from the user received via the user interface and diagnostic device data received via the diagnostic device interface.

12. The portable health monitoring device of claim 1, wherein the processor is further configured to provide feedback to the user.

13. The portable health monitoring device of claim 1, wherein the processor is configured to analyze at least a portion of the data related to the user, wherein analysis includes generating alerts, adjusting drug usage, and determining follow up questions based on the branching logic.

14. The portable health monitoring device of claim 1, wherein the data transfer interface is further configured to receive a protocol update.

15. A system for remotely monitoring healthcare patients comprising:
a portable health monitoring device, wherein the portable health monitoring device is configured to:
collect, analyze and store health information of a healthcare patient; and
remotely receive a protocol update, wherein the protocol update modifies a protocol for collecting and analyzing the health information;
a health information data store for storing the health information; and
a central monitoring server coupled to the health information data store, the central monitoring server capable of receiving health information from the portable health monitoring device, the central monitoring device further capable of analyzing the health information.

16. The system of claim 15, wherein the central monitoring server is configured to clear the health information from the portable health monitoring device.

17. The system of claim 16, wherein portable health monitoring device is configured to automatically transmit the health information to the central monitoring server.

18. A method for collecting user health information, comprising:
loading a protocol on a portable health monitoring device, wherein the protocol contains instructions for collecting and analyzing the user health information;
providing the portable health monitoring device to a user;
receiving a protocol update at the portable health monitoring device, wherein the protocol update modifies the protocol;
collecting the user health information using the portable health monitoring device; and
transmitting the user health information from the portable health monitoring device to a monitoring location.

19. The method of claim 18, further comprising:
analyzing the user health information; and
generating feedback for the user based on the user health information.

20. The method of claim 19, wherein analyzing the user health information comprises analyzing the user health information at the monitoring location.
